(19) United States
(12) Patent Application Publication
(10) Pub. No.: US 2006/0166203 A1
(43) Pub. Date: Jul. 27, 2006

(54) New sequencing method for sequencing rna molecules

(76) Inventor: Nigel Tooke, Knivsta (SE)

Correspondence Address: MINTZ LEVIN COHN FERRIS GLOVSKY & POPEO, 666 THIRD AVENUE, NEW YORK, NY, 10017, US

(21) Appl. No.: 10/529,352

(22) PCT Filed: Sep. 26, 2003
(86) PCT No.: PCT/SE03/01499

§ 371 Date: Jan. 11, 2006

Related U.S. Application Data
(60) Provisional application No. 60/413,752, filed on Sep. 27, 2002.

(30) Foreign Application Priority Data
Sep. 27, 2002 (SE) 0202867-8

(51) Int. Cl. C12Q 1/68

(52) U.S. Cl. 435/6

(57) ABSTRACT

The present invention provides a method for determination of the identity of at least one nucleotide in a RNA-molecule comprising the steps of: (i) providing the RNA-molecule, an oligonucleotide primer binding to a predetermined position of the RNA molecule, a reverse transcriptase, deoxynucleotides and other necessary reagents, in a reaction vessel; (ii) performing a primer extension reaction, whereby the oligonucleotide primer is extended on the RNA-molecule through incorporation of at least one deoxynucleotide by the action of a reverse transcriptase, resulting in the release of a PPi molecule only upon incorporation of a deoxynucleotide; and (iii) detecting the presence or absence of incorporation, thereby indicating the nucleotide identity of the RNA molecule in the relevant position. In a preferred embodiment, the sequencing of the invention is coupled to the Pyrosequencing™ reaction. A variant of the method employs incorporation of modified nucleotides, with an optionally cleavable linker arm to which is attached a label.

### TECHNICAL FIELD

The present invention relates to methods for sequencing RNA. Furthermore, the invention relates to kits for use in the methods of the invention.

### TECHNICAL BACKGROUND

The analysis of RNA has a central role in molecular biology. For example, it is increasingly recognised that single genes can encode various proteins depending on the processing of the associated mRNAs. It appears that more than half of human genes make more than one protein based on differential splicing/modifications of precursor RNAs. In addition, the sequence of various RNA molecules can be of great value in the identification of organisms, especially micro-organisms. Furthermore there is an increasing interest in the molecular biology of RNA viruses. There is therefore a clear need for effective methods for sequencing RNA.

The direct sequencing of RNAs allows researchers to analyse the transcriptome more directly than via hybridization. Various methods are available for direct sequencing of RNA (described in more detail below). These are generally based on chemical or enzymatic cleavage, or a modified version of ‘Sanger sequencing’ as used for DNA. These methods generally employ radioactivity or fluorescence for detection in combination with a separation step, typically electrophoresis. Alternatively, mass-spectrometric analysis of RNA fragments or sequence ladders has also been investigated. The more common sequencing approaches (indirect methods) require retro-transcription steps that generate cDNA molecules, which in turn may not accurately represent the messages (due to misincorporations, truncations etc.). To the inventor's knowledge, no technology today exists, which can sequence RNA directly without using radioactivity, fluorescence labelling, chemical/enzymatic degradation or a separation step. Simple, separation-independent direct sequencing of RNA would complement current chip and RT-PCR expression profiling approaches, which, when used in a screening-mode, do not differentiate between various messages generated from the same gene. In addition, a RNA sequencing method without separation step would facilitate high throughput and integration into upstream preparation steps. Some of the technologies available today are listed below in more detail.

Examples of direct analysis methods are as follows:
(1) Digestion by enzymes: Different RNases that cleave at different sites in the RNA molecule resulting in fragments that can be resolved on electrophoretic gels. The band patterns can be used to determine the sequence (Donis-Keller et al 1977).
(2) Chemical cleavage of radioactively-labelled RNA after a partial, specific modification of each kind of RNA base, followed by separation by gel electrophoresis (Peattie, 1979).
(3) Variants of ‘Sanger sequencing’ for the analysis of DNA (Sanger et al, 1977). An early example was reported (Rocca-Serra, 1984) that involved incorporation of radioactive dideoxynucleotides by a RNA-dependent DNA polymerase (AMV Reverse Transcriptase). Such methods have also been converted to fluorescent detection with fluorescent terminating nucleotides (Bauer, 1990). Sequencing of RNA using RNA-dependent RNA polymerases in combination with fluorescent chain terminators has also been reported (Makeyev and Bamford, 2001). All methods rely on separation by denaturing gel electrophoresis.
(4) Fragmentation and mass-spectrometry: This is a developing field that might enable direct sequencing depending on resolution and stability of RNA fragments (see for example U.S. Pat. No. 6,268,131 and Faulstich et al, 1997).

Common to all these methods is the need for a separation step with inherent problems of resolution, disturbances by secondary structure etc.

Indirect analysis may for example function as follows: A DNA copy of the RNA can be prepared, so-called cDNA, by annealing a DNA oligonucleotide primer to the RNA and extending the primer using a Reverse Transcriptase (RT) polymerase and deoxynucleotides. Depending on the reaction conditions the RT reaction may succeed in creating a full-length copy of the RNA. This cDNA can then be cloned into a viral or bacterial vector and can be sequenced by cycle-sequencing. Alternatively, the cDNA can be used as a template in PCR, which yields large numbers of copies of specific regions of the cDNA that can be sequenced by conventional methods of DNA sequencing.

When considering methods for sequencing RNA and DNA it is important to note the fundamental differences between these two biomolecules. The sugar portion in the nucleotides of RNA has two hydroxyl groups (-OH groups) at the 2′ and 3′ position of the ribose. The extra -OH group at the 2′ position changes both chemical and physical properties dramatically when compared to DNA, which has no hydroxyl group at the 2′ position. For example, RNA shows much higher sensitivity to degradation by sodium hydroxide, nucleases and Mg²⁺ at high pH. RNA contains no thymine, but instead contains the closely related pyrimidine uracil.

Various documents are known that disclose the sequencing of DNA, e.g. WO0043540, WO02/20836, WO02/20837, U.S. Pat. No. 4,863,849 and WO90/13666. However, none of these documents actually discloses results of the sequencing of RNA. A strategy for direct sequencing of RNA in real-time would have to solve technical problems that are not present in a DNA sequencing strategy. For example, new reagent combinations, including enzymes, buffers, salts and other additives, must be developed to ensure that step-wise primer extension is performed efficiently and accurately by an RNA-dependent enzyme capable of operating in the same environment as components required for detection (including nucleotide analogues) and without the risk of degrading RNA by chemical means, by intrinsic RNase activity of the polymerase, or by other, contaminating RNases. For example, both MMLV and AMV RT have RNase H activity in addition to pol activity. The RNase H activity competes with the pol activity for the hybrid formed between the RNA template and the DNA primer or growing cDNA strand and degrades the RNA strand of the RNA:DNA complex. RNA template that is cleaved by RNase H activity is no longer an effective substrate for cDNA synthesis, decreasing both the amount and size of the cDNA. Sequencing methods, such as sequencing-by-synthesis, based on such this would suffer from reduced read-length or signal intensity.

In addition, RNA is more prone than DNA to form complex secondary structures, which can be expected to compromise the activity of polymerase enzymes, thus demanding strategies for reduction in secondary structures or modifying the polymerase itself. It has also been reported that a significant amount of non-specific priming (so-called endogenous priming) can occur during reverse transcription regardless of what primers are included in the reaction and that this can be avoided by development of specific reagents (Ambion Inc., USA).

Thus, the research community today lacks a method for direct sequencing of RNA, which can generate high-quality data at a satisfactory throughput and effort without the complications of separation steps. Accordingly, there is a need for improved, reliable methods for sequencing RNA. The object of the invention is to provide a method for sequencing RNA, which is simple and avoids separation steps, and is thereby also amenable to scaling-up, automation and integration with sample preparation.

### SUMMARY OF THE INVENTION

This and other objects are in a first aspect of the invention accomplished by a method for determination of the identity of at least one nucleotide in a RNA-molecule comprising the steps as defined in claim 1 of the present application.

Hereby, a nucleotide sequence of a RNA molecule can be analysed in a direct way by sequencing-by-synthesis. In essence, this aspect of the invention is a development of the Pyrosequencing™ method for DNA sequences.

In another aspect of the invention, a kit for performing the nucleotide identification of the invention is provided, the kit comprising in separate vials a RNA dependent polymerase, nucleotides, necessary enzymes for a sequencing-by-synthesis reaction, and optionally other necessary reagents.

Moreover, the invention relates to a method for determining the sequence of a ribonucleic acid molecule according to claim 33. Also, the invention refers to a kit for use in this method.

### SHORT DESCRIPTION OF THE DRAWINGS

FIG. 1: Extension of a oligo (dT)₁₂₋₁₈ primer on a poly(rA) template with standard concentrations of dTTP. Single peaks are obtained after each dispensation corresponding to incorporation by the Reverse Transcriptase of one or a few nucleotides before the dTTP is consumed by apyrase.

FIG. 2: As in FIG. 1 but with a higher concentration of dTTP (added in the G position of the cassette). In this case one large peak is obtained presumably due to the complete extension of the primer along the template by the Reverse Transcriptase in the presence of large amounts of dTTP that apyrase does not fully consume before the end of the reaction. Note the scale is −10-170 relative light units.

FIG. 3: As in FIG. 1 but with dCTP as added nucleotide. The incorrect nucleotide is not incorporated by the Reverse Transcriptase and no signal is obtained.

FIG. 4: Extension of NUSPT primer annealed to the DNA oligonucleotide E3PN19 giving the expected sequence.

FIG. 5: Extension of NUSPT primer annealed to the RNA oligonucleotide E3PN19RNA giving a series of peaks that is similar to that obtained from the DNA control (FIG. 4). Severe background is seen after TCAGAC presumably due to incomplete incorporation of the nucleotides in previous steps, thus leading to a series of extended products that are out of phase. Optimisation of the relationship (nucleotide concentration : apyrase activity : reverse transcriptase activity ) dramatically reduces this problem.

FIG. 6: Extension of a oligo (dT)₁₂₋₁₈ primer on a poly(rA) template. Single peaks are obtained after each dispensation corresponding to incorporation by the Reverse Transcriptase of one or a few nucleotides before the dTTP is consumed by apyrase. Note that no incorporation is obtained after dispensing A, C or G.

FIG. 7: Klenow exo⁻-mediated extension of a DNA primer on a DNA template by Cy5-SS-dNTP.

FIG. 8: RT-mediated extension of a DNA primer on a RNA template by Cy5-SS-dNTP; signal over background for correct versus incorrect nucleotide.

FIG. 9: RT-mediated extension of a DNA primer on a RNA template by Cy5-SS-dNTP; real-time measurement of FRET-signal.

FIG. 10: Sequencing of the oligonucleotide E3PN19RNA using 60% Cy5-SS-dUTP, and 20% Cy5-SS-dCTP with a final nucleotide concentration of 2 μM. The fluorescent signals from Cy5 on the nucleotide, corrected for background, are plotted for each incorporation.

FIG. 11. Selectivity curve for Cy5-SS-dUTP. The fluorescent signal from CyS is plotted as a function of the different percentages of Cy5-SS-dUTPs in the reaction mixes.

### DEFINITIONS

By “determination of the identity of at least one nucleotide” is meant to identify the type of nucleotide, i.e A, G, C or U, that is present in the position(s) of the RNA template following directly after the 3′-end of the oligonucleotide primer binding to the RNA template. One or more nucleotides in the sequence may be determined simultaneously depending on the presence of a so-called homopolymer stretch of identical bases.

By “sequencing-by-synthesis” is meant a sequencing method as first described by Melamede, U.S. Pat. No. 4,863,849. In short, the method can be described as follows; 1) an activated nucleotide triphosphate is added to a primer-template complex; 2) the activated nucleotide is detected; 3) step 1) is repeated, whereupon the sequence can be deduced from positive incorporation of nucleotides. In this general description, the activated group can be located anywhere on the dNTP molecule; in U.S. Pat. No. 5,302,509, the activated group is attached to the sugar moiety at the 3′-position, whereas in WO 93/21340, the activated group is attached to the base. Nyren discloses a third strategy in WO 98/13523 and WO 98/28440 in which the activation is related to the detection of released pyrophosphate during the primer extension step.

By “RNA-molecule” is meant any RNA-type, such as mRNA, tRNA, rRNA, snRNA or any other kind of RNA-molecule.

By a “RNA dependent polymerase” is meant any polymerase having the ability to act on a RNA-template, such as RNA dependent DNA polymerases (otherwise known as reverse transcriptases), creating a RNA:DNA duplex, and RNA dependent RNA polymerases, creating a RNA:RNA duplex.

By “nucleotides” is in the context of the invention meant nucleotides as well as deoxynucleotides, i.e. “building blocks” for both RNA and DNA. The chemistry of any of the four nucleotides making up the RNA-strand, i.e. ATP, CTP, GTP or UTP, or any analogues thereof, as well as any of the four deoxynucleotides making up the DNA-strand, i.e. dATP, dCTP, dGTP or dTTP, or any analogue thereof is readily known by a skilled person in the art.

By a “reaction vessel” is meant any kind of reaction vial or the like, that is suitable for a RNA sequencing analysis, such as for example a microtiter plate.

As defined herein, the term “label” is meant a molecule, which is possible to detect in a suitable manner. The term “dye-label” include fluorescent molecules such as fluorescein, cyanine dyes, like Cy-3, Cy-5, Cy-7, Cy-9 disclosed in U.S. Pat. No. 5,268,486 (Waggoner et al.) or variants thereof, such as Cy3.5 and Cy5.5, but may also include molecules such as Rhodamine, BODIPY, ROX, TAMRA, R110, R6G, Joe, HEX, TET, Alexa or Texas Red.

As defined herein, the term “labeled nucleotide” or “dye-labeled nucleotide” means a nucleotide, which is connected to a label or dye-label as defined above.

The term “solid phase” is used to define an array or a carrier.

As used herein, the term “array” refers to a heterogeneous pool of nucleic acid molecules that is distributed over a support matrix. These molecules, differing in sequence, are spaced at a distance from one another sufficient to permit the identification of discrete features of the array. It may also refer to miniaturised surfaces comprising ordered immobilized oligonucleotides, DNA or RNA molecules.

As defined herein, the term “carrier” is used to represent any support for attracting, holding or binding a polynucleotide used within the fields of biotechnology or medicine. A carrier can be a carrier, such as a gel, a bead (microparticles), a surface or a fiber. Different examples of gels are acrylamide or agarose; examples of beads are solid beads, which can contain a label or a magnetic compound; beads can also be porous, such as Sepharose beads; a surface can be the surface of glass, a plastic polymer, silica or a ceramic material—these surfaces can be used to prepare so-called “arrays”. A fiber can be a starch fiber or an optical fiber and even the end of a fiber.

### DETAILED DESCRIPTION OF THE INVENTION

In a first aspect, the invention provides a method for the determination of the identity of at least one nucleotide in a RNA-molecule comprising the steps of:
(a) providing a single stranded form of the RNA-molecule;
(b) hybridising an oligonucleotide primer binding to a predetermined position of the RNA molecule;
(c) performing at least one primer extension reaction, whereby the oligonucleotide primer is extended on the RNA-molecule through incorporation of at least one nucleotide by the action of a RNA dependent polymerase;
(d) detecting the presence or absence of incorporation, thereby indicating the nucleotide identity of the RNA molecule in the relevant position.

Preferably, step (c) to (d) are repeated.

Optionally, the incorporated nucleotide(s) is (are) recorded.

In one embodiment, the presence or absence of incorporation is indicated by the presence of a detectable moiety. Also, the detectable moiety may be removed or neutralized in step (d) after the detection.

In one embodiment, step (c) is performed by including a combination of sulfurylase, luciferase and apyrase enzymes in the reaction solution, which together convert the released PPi molecule to a light signal and remove excess ATP and dNTP in preparation for incorporation of the next deoxynucleotide.

The oligonucleotide primer is a DNA or RNA oligonucleotide. The length of this primer is any length that is suitable for the purpose of the invention. However, in many cases a length in the interval of 10 to 30 nucleotides is suitable.

In one embodiment, the primer extension reaction results in the release of a residue molecule, which is detected. This residue molecule may for example be a PPi molecule, which is released only upon incorporation of a nucleotide. The detection of this PPi molecule may be performed analogous to the Pyrosequencing™ reaction for DNA.

Accordingly, in one embodiment, the detection is performed by including a luciferase enzyme, as well as other necessary enzymes, such as apyrase and sulphurylase, and reagents, such as APS and luciferin, in the reaction solution, which upon release of a PPi molecule is triggered to release light.

According to one embodiment of the invention at least one nucleotide is labelled, such as fluorescently or radioactively, thereby allowing the detection to be performed by means of detecting the presence or absence of a labelled nucleotide.

In a preferred variant of this embodiment, the label on the labelled nucleotide is cleavable.

In another embodiment, the detection is performed by means of detection of a change in physical properties of the RNA-molecule (i.e. the RNA:DNA duplex, or the RNA:RNA duplex) at incorporation of a nucleotide. For example, polarisation changes are detected, or an electronic detection system is used, or some optical changes due to nucleotide incorporation are recorded.

As said above, the RNA dependent polymerase may be an RNA dependent DNA polymerase or an RNA dependent RNA polymerase.

In case of an RNA dependent RNA polymerase, it may for example originate from any RNA virus of bacteriophage, such as bacteriophage phi 6.

In one preferred embodiment of the invention, the RNA dependent DNA polymerase is Reverse Transcriptase. The Reverse Transcriptase (RT) reaction involves extension of a DNA oligonucleotide primer on a RNA template through polymerisation of deoxynucleotides by a RT polymerase and release of pyrophosphate (PPi). It is possible to utilise this PPi in the Pyrosequencing™ enzyme cascade in the same way as the PPi released during extension of a DNA oligonucleotide primer on a DNA template by a DNA polymerase. Thus, incorporation of a correct deoxynucleotide that is complementary to a ribonucleotide in the RNA template releases a PPi molecule that leads to light release, whereas providing the RT polymerase with an incorrect deoxynucleotide would not result in an incorporation, and thus no signal. Moreover the signal will be proportional to the number of correct deoxynucleotides incorporated, thus making sequencing of homopolymer stretches possible.

In Karamoharned et al., 1998, the activity of Reverse Transcriptase is measured in a bioluminometric method involving luciferase. However, in this document no efforts of developing this activity measurement to a sequencing technology are disclosed.

The RT-reaction used in the invention has been subject to a number of problems. The invention provides the following solutions to these problems: (1) Premature termination of primer extension leading to truncated cDNA—this is typically due to low processivity of the enzyme itself and/or secondary structure in the RNA template that causes the enzyme to pause and leave the template. Common solutions to this problem include the use of thermostable RT polymerases in combination with increasing the reaction temperature, which leads to a reduction in the secondary structure of the RNA template. Additives including glycerol, methyl mercury hydroxide, methoxyamine-bisulfite and DMSO can be added to help destabilise nucleic acid duplexes and melt RNA secondary structure without inhibiting reverse trancriptases (Gibson et al. 1990; Mazo et al. 1979; Gerard, 1995). Spermidine has also been used to improve RT activity (Aoyama, 1989). If RNA amplification methods are first used then it might also be possible to modify secondary structure by incorporating rITP (see Sasaki et al 1998). In addition, the use of T4 Gene 32 Protein has been reported to reduce secondary structure in the template (Kreader, 1996; Chandler et al 1998; Villalva et al 2001) and could be included in the RT-mediated sequencing-by-synthesis reaction. Other potential solutions include the ability of retroviral nucleocapsid protein to unwind RNA (Tanchou et al, 1995), and actinomycin D can prevent hairpin loop formation during cDNA synthesis with AMV RT (Wadkins et al 2000). Additional oligonucleotides with 3′ modifications (making them non-extendable) might be used to block interfering secondary structures at specific positions.

(2) Reverse trancriptases have a tendency to terminate cDNA synthesis at homopolymer stretches of RNA (Klarman et al, 1993) that may be reduced by addition of nucleocapsid protein (DeStefano, 1995). Since the position of termination may be enzyme specific (DeStefano et al, 1991) mixes of different RT enzymes may reduce this problem.

(3) Errors in the incorporation due to misincorporation of nucleotides—RT polymerases are commonly isolated from retroviruses and have no so-called proof-reading activity (3′-5′ exonuclease). The error rate is however low and acceptable in the current invention. Indeed the lack of 3′-5′ exonuclease activity is a pre-requisite for successful sequencing-by-synthesis.

(4) Degradation of the RNA template by RNase contaminated reagents or the RNA preparation itself. This problem is generally overcome by rigorous treatment of water to be used for buffers with DEPC (diethylpyrocarbonate) to remove RNases, and also the inclusion in reaction mixes of RNase-inhibiting agents, generally recombinant proteins that bind to RNases, such as RNAGuard (Amersham Biosciences) or RNaseOUT (Invitrogen).

(5) Most RT polymerases have a RNase H activity that acts as a random endonuclease that digests RNA in RNA:DNA duplexes. This activity will naturally lead to a decrease in the amount of RNA template that can be used for primer extension. RT polymerases with low RNase H activity (M-MuLV), and mutants that completely lack this RNase H activity are now available (for example Thermoscript RT and Superscript II from Invitrogen Corporation; see also Gerard et al 2002).

(6) Reverse transcription products may be generated even without primers, so called endogenous priming. Such problems may be due to contaminating tRNA(Agranovsky 1992) and been overcome using Endo free Reverse transcriptase (Ambion).

The RT-polymerase of the invention is for example chosen from the group comprising: HIV-1 RT, M-MuLV RT, AMV RT, RAV2 RT, Thermoscript AMV RT, Superscript II M-MuLV RT. Also included in the scope of the invention are any other RT enzymes meeting the demands of the invention as specified below in this application, including Tth DNA polymerase in the presence of Mn²⁺ ions.

In one embodiment of the invention a mixture of RNA dependent polymerases is added to the reaction mixture of step (a). Hereby several properties, specific for various polymerases, may be combined.

RT enzymes are commonly used at high temperatures (37° C.-55° C.) and at a pH of 8,3-8,4. This is in direct contrast to the Pyrosequencing™ reaction that is carried out at 28° C. and a pH of 7,6. It should be noted, however, that the optimal conditions for RT have been chosen to ensure high processivity and extension of the cDNA product over distances of several thousand bases, whereas direct RNA sequencing by RT-mediated sequencing-by-synthesis analyses would demand only extension with 10-100 bases. Thus sub-optimal conditions for RT are in some cases acceptable. Optimisation of the reaction conditions to suit all components in the cascade is possible. Also, some polymerases are thermostable and allow higher temperatures.

Accordingly, in one embodiment the extension reaction is performed at a temperature ranging from 28 to 70° C.

In another embodiment, the pH of the extension reaction solution is in the interval from 7.6 to 8.6, preferably from 8.0 to 8.4.

Deoxynucleotide concentrations used in RT reactions are generally in the range of 0,5-1 mM. In contrast, a Pyrosequencing™ reaction involves low micro molar concentrations that may improve the fidelity of the reaction by reducing the risk of misincorporation. However, HIV, M-MLV and AMV RT have average processivities of 50-100 nucleotides at dNTP concentrations in the range 25-150 μM (≧K_{m dNTP}). M-MLV RT processivity at 25 μM dNTP is approximately 70 nt. At 500 μM processivity for H⁻ and H⁻ M-MLV is 30 nt. An additional subject of optimisation is the balance between supplying the polymerase with deoxynucleotide at a sufficient concentration, and the activity of apyrase that is used to degrade the current deoxynucleotide in preparation for the dispensation of the next deoxynucleotide.

Thus, in one embodiment, the concentration of deoxynucleotides is in the interval from 1 μM to 1 mM.

In order for the reaction of the invention to work properly, a salt is preferably added to the reaction mixture. The positive ion in this salt is preferably a monovalent metal ion, such as Li, K or Na. The negative ion of this salt is preferably an acetate ion, Ac⁻. The concentration of the salt in the reaction mixture is preferably in the interval from 10 to 100 mM.

One deoxynucleotide, dATP, functions as a substrate for luciferase in the Pyrosequencing™ reaction and will therefore give a background signal. The solution to this problem has been to exchange dATP for an analogue, alpha-S-dATP that the DNA polymerase can incorporate into the extended primer, but that luciferase cannot use as a substrate. The challenge in the RT-mediated Pyrosequencing™ reaction is to identify an RT polymerase capable of incorporating such analogues. Indeed data presented here shows that RT can incorporate alpha-S-dATP. Alternative approaches include acceptance of the background from dATP but with software-correction of the signal, and/or the use of a mutant form of luciferase that cannot utilise dATP as a substrate.

Accordingly, in one embodiment, the deoxynucleotide dATP is exchanged for the analogue alpha-S-dATP.

When a RNA dependent RNA polymerase is used, the nucleotide ATP is in accordance with the discussion above exchanged for the analogue alpha-S-ATP (or alpha-S-rATP).

In yet another embodiment, the luciferase enzyme is in a mutant form, which is unable to utilise dATP as a substrate.

The high level of secondary structure of the RNA template can cause premature truncation of the extending cDNA strand and is generally overcome through an increase in reaction temperature and, where possible, the use of thermostable enzymes. Simple additives such as glycerol, methyl mercury hydroxide, methoxyamine-bisulfite, spermidine or DMSO can be added to destabilise nucleic acid duplexes and melt RNA secondary structure. Alternatively rITP can be incorporated when amplifying an RNA molecule to be analysed. In addition, the use of T4 Gene 32 Protein has been reported to reduce secondary structure in the template and can be included in the RT-mediated sequencing-by-synthesis reaction. Other solutions include the ability of retroviral nucleocapsid protein to unwind RNA, and the ability of actinomycin D to prevent hairpin loop formation during cDNA synthesis with AMV RT. Another alternative is to cleave the RNA with a specific RNase such that the complexity of the secondary structure is reduced, and then isolate and sequence the fragment of interest. Additional oligonucleotides with 3′ modifications (making them non-extendable) might be used to block interfering structures at specific points. Also, SSB (single stranded binding protein), formamide, glycerol and a blocking primer may be used.

Accordingly, in one embodiment, at least one RNA-secondary structure reducing reagent, preferably chosen from the group comprising glycerol, methyl mercury hydroxide, methoxyamine-bisulfite, spermidine, DMSO, incorporation of rITP (or other rNTP analogue), T4 Gene 32 Protein, retroviral nucleocapsid protein and actinomycin D, blocking oligonucleotide, SSB, formamide is included in the extension reaction.

The luciferase in the Pyrosequencing™ reaction is sensitive to Cl⁻ ions and this ion is generally replaced by acetate ions when preparing buffers. Certain RT polymerases are reportedly capable of operating in these conditions, for example ThermoScript RNase H⁻ Reverse Transcriptase (Invitrogen Corporation, USA).

It is possible to amplify RNA by a number of methods (for review see Chan and Fox, 1999). These include the isothermal methods Nucleic Acid Sequence-Based Amplification (NASBA; see Compton, 1991, and Kievits et al 1991), Transcription-Mediated Amplification (TMA; Hill, 1996), and Self-Sustained Sequence Replication (3SR, Guatelli et al 1990). Methods such as TMA that are based on RNA transcription can also be used to prepare multiple copies of RNA from a DNA target sequence .All will, of course, yield template suitable for further analysis e.g. sequencing. Indeed the use of such amplification methods isof great benefit in providing large quantities of high-quality template for analysis.

Accordingly, in one embodiment, the RNA molecule is subjected to a RNA amplification prior to the extension reaction. Also, GTP may be exchanged for ITP in this reaction, as discussed above.

Most RT polymerases have a RNase H activity and acts as a random endonuclease that digests RNA in RNA:DNA duplexes. This activity will naturally lead to a decrease in the amount of RNA template that can be used for primer extension. RT polymerases with low RNase H activity, and even mutants that completely lack this RNase H activity are now available (e.g. ThermoScript RNase H⁻ Reverse Transcriptase and SuperScript II RNase H Reverse Transcriptase (Invitrogen Corporation, USA). In addition, Tth DNA polymerase has a very efficient intrinsic reverse transcriptase activity in the presence of Mn²⁺ ions and lacks RNase H activity (Loeb et al, 1973; Myers and Gelfand, 1998).

In yet another embodiment, the RT-polymerase essentially lacks RNase H activity. By “essentially lacks” is in the context of the invention meant a RNase H activity lower than 1.0%, and preferably equal to or lower than 0.5%.

The complexity of the RNA population in an isolate leads to challenges in terms of specificity of priming. The DNA oligonucleotide primer will most commonly have a sequence designed to anneal only to the region of interest. This level of specificity can be enhanced by prior amplification of the RNA using various methods involving additional, region-specific primers, or by isolating and purifying the RNA of interest using oligonucleotides immobilised on a solid-phase. Indeed the oligonucleotide primer may itself be immobilised on a solid-phase, such as a biotin-streptavidin or biotin-avidin system or covalent immobilisation before or after annealing to the RNA molecule to be analysed. Thus, a solid-phase facilitates sequencing in complex mixtures, and also changes in buffer composition if RNA amplification is first used to prepare sufficient template for sequencing. The solid phase method is based on (1) immobilised oligonucleotide for capture of a specific template, and a separate sequencing primer, or (2) immobilised sequencing primer.

In still another embodiment, the oligonucleotide primer is immobilised to a solid phase.

In a further embodiment, the quantity of the RNA-molecule is determined by measuring the intensity of the incorporation signal and comparing it to a reference. Hereby, the method of the invention may be used for quantitative purposes, i.e. to analyse the quantity of RNA-template in a sample.

In a second aspect, the invention refers to a kit for performing the nucleotide identification of the invention, comprising in separate vials a RNA dependent polymerase, nucleotides, necessary enzymes for a Pyrosequencing™ reaction, and optionally other necessary reagents. Hereby, a kit is provided comprising necessary components and reagents for performing the method of the invention.

In another embodiment, the kit further comprises a RNA quantity reference sample. Hereby, the kit may be used for quantification purposes, i.e. to analyse the quantity of RNA in a sample of interest.

In another aspect, the invention relates to a method for determining the sequence of a ribonucleic acid molecule comprising the steps of;
a) providing a single-stranded form of said ribonucleic acid molecule;
b) hybridizing a primer to said single stranded form of said ribonucleic acid molecule to form a template/primer complex;
c) enzymatically extending the primer by the addition of an RNA dependent polymerase and a mixture of nucleotides and a derivative of said nucleotides, wherein the derivative of said nucleotide comprises a label linked to a nucleotide via an optionally cleavable link and wherein the proportion in the mixture between the nucleotides and the derivative of said nucleotide is within the range of 1-60%,1-50%, 1-40%, 1-30%, or 1-20%, preferably in the range of 5-60%, 5-50%, 5-40%, 5-30%, or 5-20%, or more preferably in the range of 10-60%, 10-50%, 10-40%, 10-30%, or 10-20%
d) determining the type of nucleotide added to the primer;

In one embodiment, steps c) to d) above are repeated at least once.

The reason for using mixtures of nucleotides versus derivative of said nucleotides, is that two phenomena can occur in a reaction according to this aspect of the invention, which phenomena make the dilution of labelled (detectable) nucleotides with natural nucleotides preferable.

For the first, fluorescent quenching occurs when several nucleotides are incorporated due to homopolymer stretches in the template. Secondly, spontaneous cleavage of the S-S-bond can occur in incorporated labelled nucleotides that are in proximity to a previously incorporated and cleaved labelled nucleotide bearing a free thiol group. These two problems are solved by diluting the labelled nucleotide with natural nucleotide, therby reducing the probability that there are neighbouring labelled/cleaved nucleotides on individual extended primer molecules.

The polymerase enzymes (such as DNA polymerases and Reverse Transcriptases) exhibit a selectivity of natural nucleotides over labelled nucleotides that can differ between enzymes and between nucleotide bases. Hence, the optimum mixtures will vary between nucleotide bases and between enzymes. This explains the use of different mixes in the examples 5-6 below.

In a further embodiment, the label is neutralized after step d) by the addition of a label-interacting agent or by bleaching, preferably by photo-bleaching. The label can be neutralized by bleaching (photo bleaching) or by adding a compound that neutralizes the emitted fluorescence, such as another label, then reducing the emitted light by quenching.

In certain embodiments it is preferable to cleave off the label from the nucleotide. This is made possible by using a linker between the nucleotide and label that is cleavable by e.g. a reducing agent. Thus, a method according to the above is provided, in which the link between the incorporated nucleotide and the label is cleaved after step d). According to this, a method according to the above is provided, in which the link between the fluorophore and nucleotide is an S-S bridge.

In one embodiment the cleavage is performed by the addition of a reducing agent, thereby exposing a thiol group.

In one embodiment the exposed thiol group is capped with a suitable reagent such as iodoacetamide or N-ethylmaleimide.

The object of this aspect of the invention may be met by using a linker that is short enough to prevent interaction between adjacent labels. According to this, the length of the linker between the disulfide bridge and the base of the nucleotide is preferably shorter than 8 atoms. Thus, in a further embodiment the linker between the disulfide bridge and the base is shorter than 8 atoms.

In one embodiment step c) is performed at a pH 7.6 to 8.6, preferably from pH 8.0 to 8.4.

In a further embodiment the derivative of said nucleotide is a dideoxynucleotide or an acyclic nucleotide analogue.

In yet a further embodiment, an agent chosen from the group comprising the following; alkaline phosphatase, PP-ase, apyrase, dimethylsulfoxide, polyethylene glycol, polyvinylpyrollidone, spermidine, detergents such as NP-40, Tween 20 and Triton X-100 is added.

In one variant of this aspect of the invention, a mixture of natural nucleotides and a derivative of said nucleotides is provided, wherein the derivative of said nucleotides comprises a label linked to a nucleotide via an optionally cleavable link and wherein the proportion in the mixture between the nucleotides and the derivative of said nucleotides is within the range of 1-60%, 1-50%, 1-40%, 1-30%, or 1-20%, a preferred proportion is in the range of 5-20%, 5-30%, 5-40%, 5-50% or 5-60%, and even more preferred in the range of 10-20%, 10-30%, 10-40%, 10-50% or 10-60%.

A further variant of this aspect of the invention is a kit which comprises, in separate compartments; a mixture according to previously mentioned aspects, and at least one of the following components; an RNA dependent polymerase, a reducing agent, a carrier, a capping agent, an apyrase, an alkaline phosphatase, a PP-ase, a single strand binding protein or the protein of Gene 32, for performing the method according to any of the steps in the above-mentioned methods.

The invention also relates to a kit that contains suitable reagents for performing the method of the invention.

Consequently, a further embodiment is a kit which comprises, in separate compartments, at least two of the following components; mixture of labeled and non-labeled nucleoside triphosphates, RNA dependent polymerase, reducing agent, carrier, capping agent, apyrase, single strand binding protein, for performing the method according to any of the above-mentioned embodiments.

This approach to sequence has been shown for DNA, see example 3 (comparative), and for RNA, see example 4-6.

RNA and DNA oligonucleotides are readily commercially available and can be ordered from SGS (Sweden) and Dharmicon (USA).

RNases must be eliminated/inactivated by treatment of reagents (and even plastics) using DEPC. RNAguard (or similar reagents) can be used to protect RNA during the assay.

In table 1, optimal conditions for some RT enzymes used in the invention are shown.

TABLE 1
Optimal conditions for some RT enzymes

| Company | Enzyme | Rnase H | pH | K/NaCl*** | MgCl₂ | DTT | dNTP**** | Temp |
|---|---|---|---|---|---|---|---|---|
| Amersham | AMV | Y | 8.3 | 25 | 8 | 1 | | 42 |
| Biosciences | M-MuLV | Low | 8.3 | 75 | 3 | 10 | | 37 |
| | HIV-1 | Y | 8.3 | 50 | 10 | 3 | 0.5 | 37 |
| | RAV2 | Y | 8.3 | 75* | 3 | 10 | | 37 |
| Invitrogen | Thermoscript AMV | <0.5% | 8.4 | 75 | 8 | 5 | 1 | >50 |
| Corp. | | | | KAc | MgAc | | | |
| | Superscript II | <0.5% | 8.3 | 75** | 3 | 10 | 0.5 | <50 |
| | M-MuLV*** | | | | | | | |
| | AMV | Y | 8.3 | 50 | 10 | 10 | 0.5 | 42 |

*RNA-dependent DNA pol: 50-100 mM
*DNA-dependent DNA-pol: 10-20 mM
**<50 mM reduces activity to 75% of maximum.
***Exchange of Cl⁻ by Ac⁻ ions possible.
****dNTP concentration and processivity

HIV, M-MLV and AMV RT have average processivities of 50-100 nucleotides at DNTP concentrations in the range 25-150 μM (≧K_{m dNTP}). M-MLV RT processivity at 25 μM dNTP is approximately 70 nt. At 500 μM processivity for H− and H+ M-MLV is 30 nt.

The basis of the Pyrosequencing™-reaction, which is referred to herein, is as follows: Themethod was developed at the Royal Institute of Technology in Stockholm (Ronaghi et al.,1998, Alderbom et al.,2000), and isbased on “sequencing by synthesis” in which the deoxynucleotides are added one by one during the sequencing reaction. An automated sequencer, the PSQ96™ instrument, has recently been launched by Pyrosequencing AB (Uppsala, Sweden). The principle of the Pyrosequencing™ reaction for RNA: A single stranded RNA fragment (optionally attached to a solid support), carrying an annealed DNA (optionally an RNA) sequencing primer acts as a template for the Pyrosequencing™ reaction. In the first two dispensations, substrate and enzyme mixes are added to the template. The enzyme mix consists of four different enzymes; RNA-dependent polymerase, such as reverse transcriptase (optionally a mix of reverse transcriptases), ATP-Sulfurylase, Luciferase and Apyrase. The nucleotide triphosphates are added sequentially according to a specified order dependent on the template and determined by the user. If the added nucleotide triphosphate matches the template, the RT polymerase will incorporate it into the growing DNA(RNA)/RNA-duplex. By this action, pyrophosphate, PPᵢ, will be released. The ATP-Sulfurylase converts the PPi into ATP, and the third enzyme, Luciferase, transforms the ATP into a light signal. Following these reactions, the fourth enzyme, Apyrase, will degrade the excess deoxynucleotides and ATP, and the template will at that point be ready for the next reaction cycle, i.e. another nucleotide triphosphate addition. Luciferin and APS are substrates for the reaction. Since no PPi is released unless a deoxynucleotide is incorporated, a light signal will be produced only when the correct nucleotide is incorporated. The software steering the PSQ 96 system will present the results as peaks in a pyrogram™, where the height of the peaks corresponds to the number of deoxynucleotides incorporated. An advantage with sequencing-by-synthesis is that the first base directly after the extension primer can be read with high accuracy.

A potential problem, which has previously been seen with sequencing-by-synthesis methods, is that false signals may be generated and homopolymeric stretches (i.e. CCC) are difficult to sequence with accuracy. This may be overcome by the addition of a single-stranded nucleic acid binding protein (SSB) once the extension primers have been annealed to the template nucleic acid. The use of SSB in sequencing-by-synthesis is disclosed in WO00/43540 of Pyrosequencing AB.

An additional method for sequencing-by-synthesis of RNA that is presented here is based on the use of labelled nucleotides. Previous work has shown that nucleotides, to which are attached a fluorescent group by a cleavable linker arm (for example a disulfide bridge), can be used by DNA polymerase to extend a DNA oligonucleotide annealed to a DNA template. WO 00/53812 and WO 00/50642 describe the use of a nucleotide where a disulfide-containing linker is used for coupling a dye to the nucleotide. This enables easy removal of the dye by redox cycling. In WO 00/53812 the dye is linked to the base (only dCTP is described) and in WO 00/50642 the dye is attached to the 3′-position of the sugar moiety. U.S. Pat. No. 6,613,523 also describes a method involving cleavable tags attached to the 3′-position.

In the method presented here a reverse transcriptase or other RNA-dependent polymerase is used to incorporate a mixture of labelled and non-labelled nucleotides onto the DNA primer annealed to a RNA template. Unincorporated nucleotide is removed and the fluorescence of any incorporated nucleotides is measured. The fluorescent label is then cleaved from the incorporated labelled nucleotides by a reducing agent, such as dithiothreitol. The process can then be repeated with other nucleotides to determine the sequence of the template. The labelled nucleotides are diluted with unlabelled nucleotides to avoid fluorescent quenching and also chemical interactions between the free thiol groups of cleaved, incorporated nucleotides, and neighbouring uncleaved labelled nucleotides, as described elsewhere in this document.

The invention will now be described with reference to the following examples. However, these examples are only intended to exemplify the invention, and do not limit the scope of the invention.

### EXAMPLES

### Example 1

All reagents and consumables were prepared to minimise the risk of RNase contamination.

The following were mixed in the well of a PSQ96 Plate:

| | μL |
|---|---|
| Reverse Transcriptase Buffer (5× concentration)* | 10 |
| Poly(rA)*oligo(dT)₁₂₋₁₈ (approx. 10 μM) | 1 |
| DTT 0.1 M | 4 |
| RNaseOUT (Invitrogen) 40 U/μL | 2 |
| SuperScript II RNase H⁻Reverse Transcriptase | 1 |
| (Invitrogen) 200 U/μL | |
| Water | 22 |

*250 mM Tris acetate (pH 8.4 at room temperature), 375 mM potassium acetate, 40 mM magnesium acetate.

The plate was then placed in a PSQ96 Instrument that dispensed automatically Enzyme Mix minus DNA polymerase (i.e. Sulphurylase, Luciferase and Apyrase) and Substrate (APS and luciferin) mixes followed by nucleotides. The nucleotides were (1) a standard concentration of dTTP giving a final concentration in the well of 2.2 μM immediately after each dispensation, (2) a 50× concentrated dTTP giving a final concentration in the well of 100 μM immediately after each dispensation, and (3) a standard concentration of dCTP giving a final concentration in the well of 1.8 μM immediately after each dispensation.

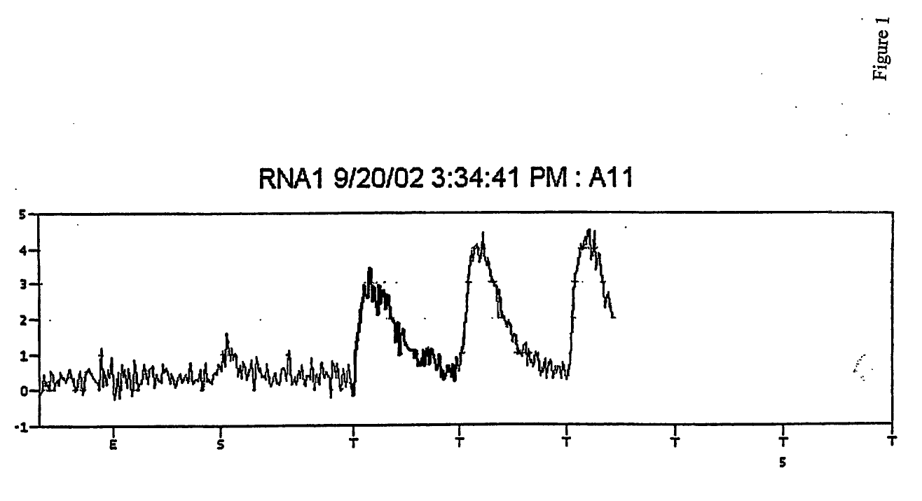

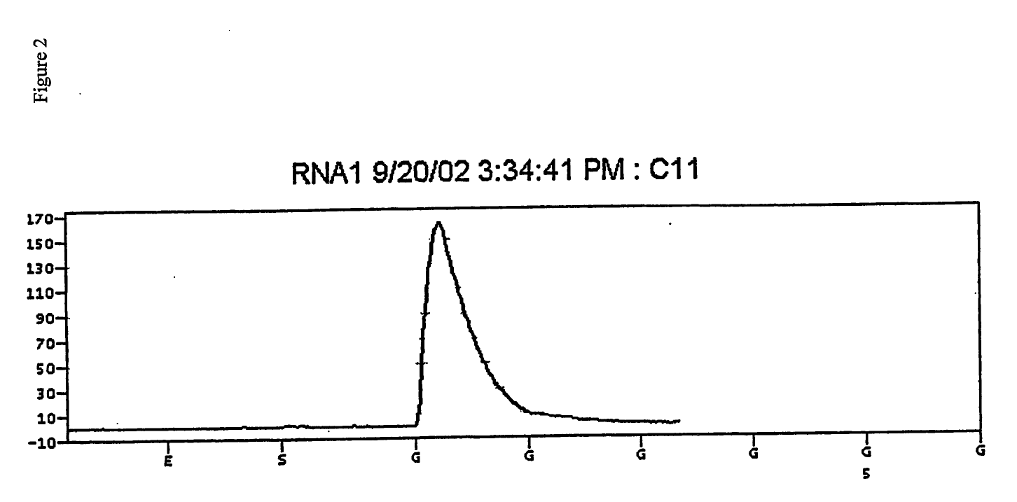

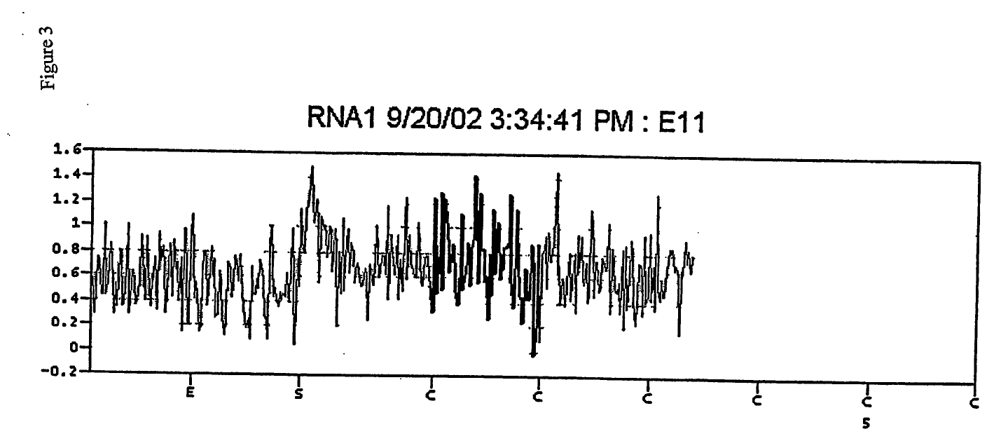

The results of the experiment are shown in FIGS. 1, 2 and 3.

### Example 2

All reagents and consumables were prepared to minimise the risk of RNase contamination.

The following templates were prepared:

(1) A DNA control consisting of 10 pmoles E3PN19 to which an excess of 30 pmoles NUSPT primer was annealed by incubating in 200 μL Annealing Buffer (20 mM Tris-acetate, pH 7.7, 5 mM magnesium acetate) at 65° C. for 5 minutes and then cooling to room temperature. Forty microlitres (2 pmoles) of this was used in the control well.

(2) A RNA test template consisting of 100 pmoles E3PN19RNA, an RNA with the same sequence as E3PN19b, to which an excess of 300 pmol NUSPT primer was annealed by incubating in 200 μL water at 65° C. for 5 minutes and then cooling to room temperature. Twenty microlitres (10 pmoles of template) of this was used in the test well.

The sequences of the E3PN19 and NUSPT oligonucleotides are shown below.

| | |
|---|---|
| E3PN19 | CTGGAATTCGTCTGAACTGGCCGTCGTTTTACAAC |
| E3PN19RNA | CUGGAAUUCGUCUGAACUGGCCGUCGUUUUACAAC |
| NUSPT | GTAAAACGACGGCCAGT |

When combined E3PN19 or E3PN19RNA give a duplex with NUSPT such that the extension of the primer will give the following sequence:

TCAGACGAATTCCAGC

(3) A RNA/DNA duplex consisting of oligo (dT)₁₂₋₁₈ annealed to poly (rA) (Amersham Biosciences). Approximately 10 pmoles of this was used in the test well.

The wells were prepared according to the table below, made up to 40 μL with water.

| | | | | | Klenow | |
|---|---|---|---|---|---|---|
| | | | | | DNA | |
| | | | | | Poly- | RT* |
| | | RNase | | | merase | 200 |
| | Buffer | inhibitor | 0.1 M | | exo- 10 | U/μL) |
| No | (1) | (2) | DTT | Template | U/μL | (3) |
| 1 | — | — | — | 2 pmoles | 1 | |
| | | | | E3PN19bDNA | | |
| 2 | 10 μL | 2 | 4 | 10 pmoles | | 1 |
| | RT Buffer | | | E3PN19RNA | | |
| 3 | 10 μL | 2 | 4 | 10 pmoles | | 1 |
| | RT Buffer | | | Poly(rA)*oligo | | |
| | | | | (dT)₁₂₋₁₈ | | |

(1) 250 mM Tris acetate (pH 8.4 at room temperature), 375 mM potassium acetate, 40 mM magnesium acetate
(2) RNaseOUT Ribonuclease Inhibitor (Invitrogen Corporation) 40 U/μL
(3) SuperScript II RNase H⁻ Reverse Transcriptase (Invitrogen Corporation) 200 U/μL

Pyrosequencing™ reagents were standard products except that Klenow DNA polymerase exo− was omitted from the Enzyme Solution.

The plate was then placed in a PSQ96 Instrument that dispensed automatically Enzyme Mix minus DNA polymerase (i.e. Sulphurylase, Luciferase and Apyrase) and Substrate (APS and luciferin) mixes followed by nucleotides. The nucleotides dispensed in a cyclic fashion in the order CTAG.

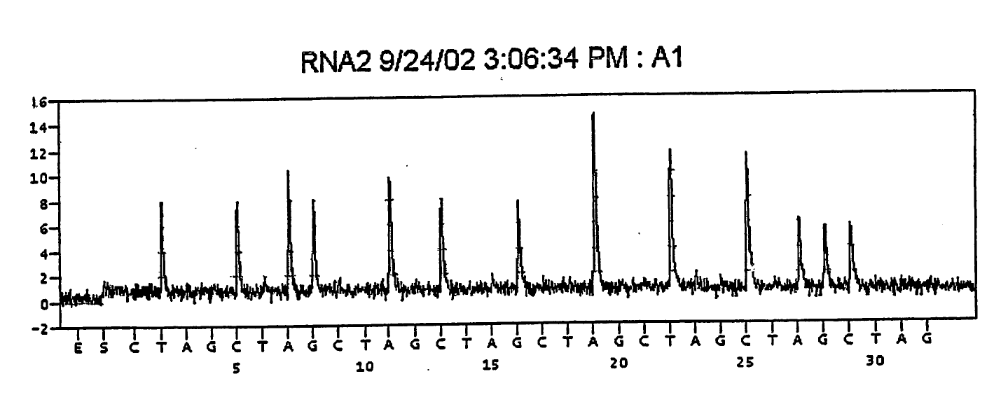

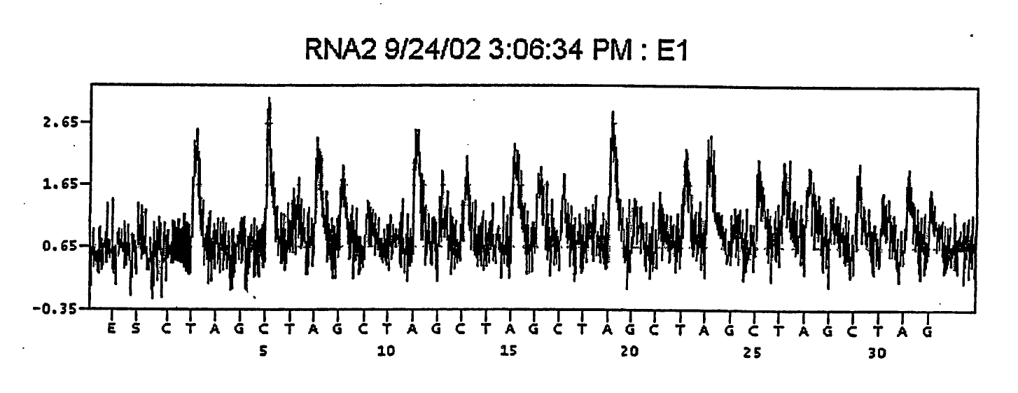

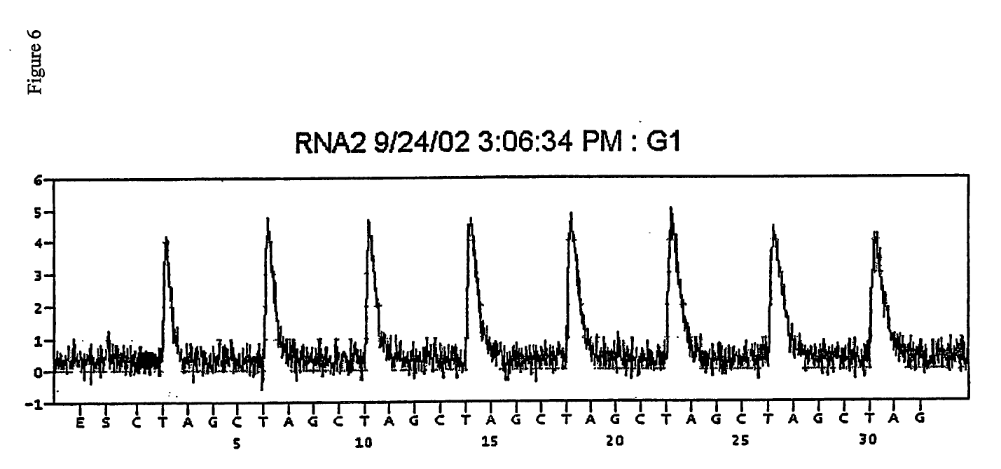

The results are shown in FIGS. 4-6.

### Example 3

### Example: Sequencing, Using “Directed Dispensation”, of the Oligonucleotide E3PN19b

The bases to be incorporated are indicated in bold.

NUSPT: fluorescein-GTAAAACGACGGCCAGTUCAGACGAA
E3PN19b CAACATTTTGCTGCCGGTCAAGTCTGCTTAAGGTCG-
biotin

Five pmole of template E3PN19b and 3 pmole primer NUSPT-FL were annealed at 80° C. for five minutes in 25 μl Annealing Buffer (20 mM Tris-acetate, 5 mM MgAc₂, pH 7.6). After cooling to room temperature, the template was bound to streptavidin beads by adding 4 μl bead slurry (Streptavidin Sepharose High Performance beads) together with 29 μl Binding buffer (10 mM Tris-HCl, 2 M NaCl, 1 mM EDTA, 0.1% Tween-20) followed by incubation at room temperature for 20 min with shaking at 1400 rpm.

The beads were transferred to a filter plate (Multiscreen, Millipore) and washed four times with 2×AB (40 mM Tris-acetate, 10 mM MgAc₂, pH 7.6). The filter plate was pre-warmed at 37° C. for 2 minutes. The first base was incorporated by adding 50 rated μL Reaction Mixture (0.5 μM Cy5-SS-dUTP, 0.5 μM dUTP, 5 U Klenow exo⁻, 2×AB) and incubating at 37° C. for 2 minutes.

The beads in the wells of the filter plate were washed four times with TENT ( 40 mM Tris-HCl pH 8.8, 50 mM NaCl, 1 mM EDTA, 0.1% Tween 20) under vacuum. The beads were resuspended in 50 μl TENT and transferred to a fluorimeter plate to a fluorimeter plate to measure the fluorescence of the CyS-labelled nucleotide (excitation 590 nm, emission 670 nm) and the fluorescence of the fluorescein-labeled primer (excitation 485 nm, emission 535 nm) using a fluorimeter (Victor2, Perkin-Elmer). The fluorescein signal was used to normalize results for variation in transfer of beads. After measuring, the beads were transferred back into the filter plate and the Cy5-label was cleaved from the incorporated dUTP by incubation with Cleavage Buffer (250 mM dithiothreitol, 50 mM NaCl, 40 mM Tris-HCl, 20 mM MgCl₂, pH 8.4) for 3 minutes at 37° C. The beads were then washed two times in TENT and two times in 2×AB.

Subsequent Cy5-SS-dNTPs were incorporated in the same manner as the first and cleaved as described above. The sequencing reaction mixes were the same for all four deoxynucleotides except for the proportion of labeled dNTPs. The mixes contained 20% Cy5-SS-dCTP, 30% Cy5-SS-dATP or 30% Cy5-SS-dGTP with the balance made up with the corresponding natural deoxynucleotide.

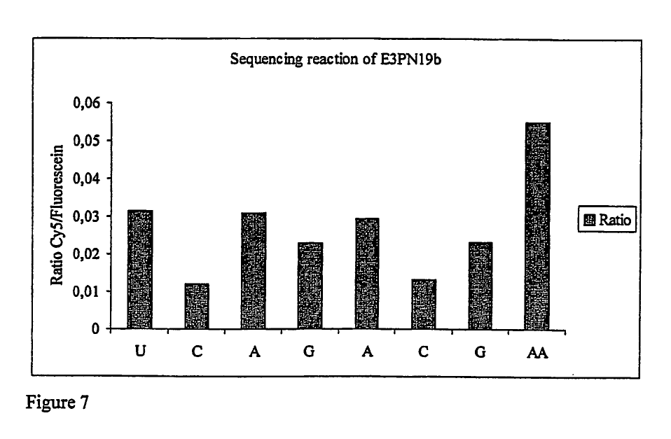

As can be seen in FIG. 7, the signals obtained were reproducible and stable throughout the sequence for the different nucleotides. The internal variation in signal height between different bases was due to differences in the way Klenow exo⁻ polymerase accepts the labeled nucleotides. The level of incorporated of nucleotides was checked by analyzing the immobilized templates by using a PSQ 96 system and associated kits according to the manufacturers instructions (Pyrosequencing AB, Sweden) such that the absence of a peak at the point of dispensing respective dNTPs was indication of complete incorporation in the foregoing experiment. All incubations gave better than 95% incorporation as assessed by the curves in a pyrogram (results not shown).

Based on these finding and by replacing the Klenow exo⁻ with an RNA dependent polymerase such as a Reverse Transcriptase or more preferably SuperScript II RNase H⁻ Reverse Transcriptase an RNA template can be sequenced and a similar result is expected.

### Example 4

### Reverse Transcriptase-Mediated Extension of a DNA Primer on a RNA Template by Cy5-SS-dNTP

The sequences (5′→3′) of the E3PN19RNA and fluorescein-labelled (FL) NUSPT oligonucleotides used in these experiments are shown below with the position of the primer site on the template underlined.

E3PN19 (RNA) CUGGAAUUCGUCUGAACUGGCCGUCGUUUUACAAC
FL-NUSPT (DNA) FL-GTAAAACGACGGCCAGT

A:

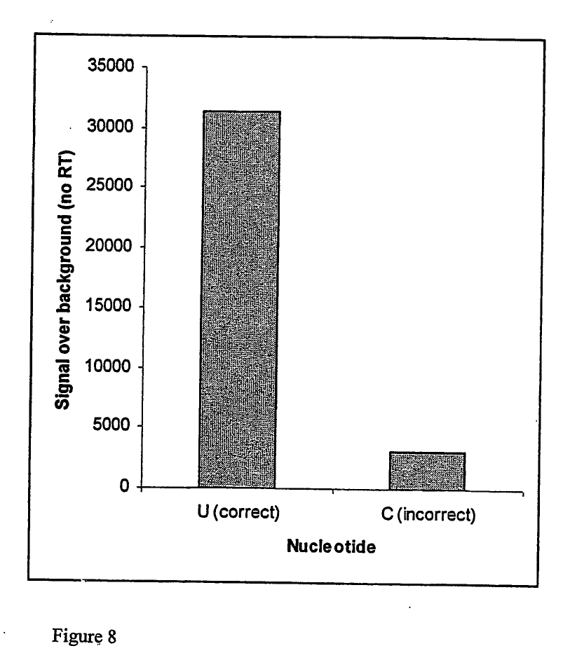

Five picomoles of the RNA template, E3PN19RNA, and 15 pmol of the complementary fluorescein-labelled DNA primer, FL-NUSPT (see above) were annealed in 5 μL water by incubating at 65° C. for 5 minutes and then cooling to room temperature. Components of the reverse-transcriptase reaction mix were then added to give a total volume of 50 μL: 10 μL 5× reaction buffer (250 mM Tris-HCl, pH 8.3, 375 mM KCl, 15 mM MgCl₂; Invitrogen Corp.), 40 U RNaseOUT recombinant ribonuclease inhibitor (Invitrogen Corp.), 200 U Superscript II RNase H⁻ Reverse Transcriptase (Invitrogen Corp.), and 50 pmol of Cy5-SS-dUTP or Cy5-SS-dCTP. Controls without RT were included. The reactions were incubated at 37° C. for 5 minutes. The level of Cy5-SS-dNTP incorporated was measured by Fluorescence Resonance Energy Transfer (FRET). Measurements were performed in a fluorimeter (Victor2, Perkin-Elmer) by exciting the fluorescein on the primer at 485 nm and measuring the resonance transfer signal from any Cy5 incorporated onto the primer at 670 mn, the emission wavelength for Cy5. The results are shown in FIG. 8 and clearly show that the correct nucleotide (U) gave a signal over background (absence of RT) whilst the incorrect nucleotide (C) did not.

B:

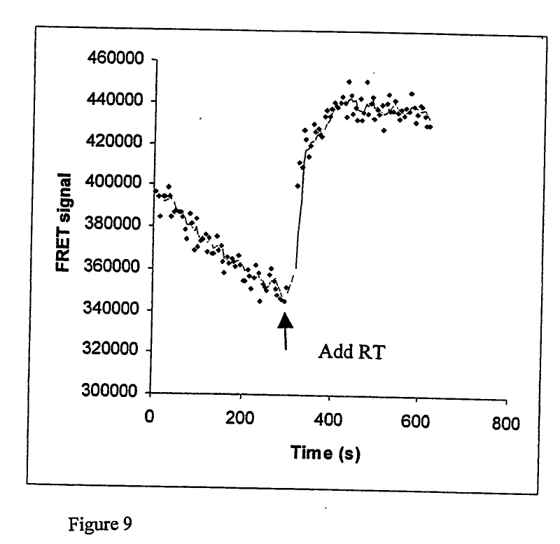

The activity of reverse transcriptase in incorporating Cy5-SS-dUTP into the primer/template FL-NUSPT/E3PN19RNA was determined in real-time. The primer and template were annealed and mixed with reaction components as in Experiment 1 but reverse transcriptase was omitted. The FRET signal was measured in real-time in a fluorimeter (Victor2, Perkin-Elmer) with an excitation wavelength of 485 nm and an emission wavelength of 670 nm. The reaction was then started by adding reverse transcriptase (200 U Superscript II RNase H⁻ Reverse Transcriptase in 1 μL). The results are shown in FIG. 9 and clearly show an increase in FRET signal on the addition of the enzyme, thus demonstrating the incorporation of Cy5-labelled nucleotide into the primer/template complex.

### Example 5

### Sequencing RNA Using Cleavable Nucleotides

Reagents were treated with diethylpyrocarbonate, RNaseZap (Ambion) or RNAse-cure (Invitrogen) to remove RNases where necessary.

The sequences (5′→3′) of the E3PN19RNA and NUSPT oligonucleotides are shown below with the position of the primer site on the template underlined.

| | |
|---|---|
| E3PN19RNA | CUGGAAUUCGUCUGAACUGGCCGUCGUUUUACAAC |
| NUSPT-B | Biotin-GTAAAACGACGGCCAGT |

When combined E3PN19RNA gives a duplex with NUSPT such that the extension of the primer will give the following initial sequence: TC

The biotinylated oligonucleotide primer NUSPT was annealed to the RNA oligonucleotide template E3PN19RNA by incubating 40 pmole (2 pmole per replicate) NUSPT-B with 120 pmole E3PN19RNA (6 pmole per replicate) in 400 μL Annealing Buffer (20 mM Tris-acetate, 5 mM MgAc₂, pH 7.6) at 60° C. for 5 minutes and cooled to room temperature. The biotinylated primer annealed to the template was then captured on a solid-phase by incubating with 500 μL Binding Buffer (10 mM Tris-HCl, pH 7.6, 2 M NaCl, 1 mM EDTA, 0.1% Tween 20) and 80 μL Streptavidin Sepharose High Performance (Amersham Biosciences) and shaking for 20 minutes. The beads were then washed 4 times with 400 μL TE (10 mM Tris, 1 mM EDTA, pH 8.0) in filter tubes (Nanosep MF GHP 0.45 μm, Pall), resuspended in 500 μL TE and 25 μL aliquots (corresponding to 2 pmole NUSPT-B:E3PN19RNA) were transferred to the wells of a filter plate (MultiScreen; Millipore) and drained by applying vacuum. Fifty microlitres of Reaction mixes were added as described below and the plate was incubated at 37° C. for 5 minutes followed by washing with 4×100 μL Washing Buffer (TE containing 50 mM NaCl and 0.1% Tween 20) and 3×400 μL TE. When the cycle of treatments was completed, the beads were resuspended in 2×100 μL TE and transferred to a fluorimeter plate (ThermoLabsystems). Fluorescence was measured in a Victor² Multilabel Counter with an excitation of 590 nm and emission of 670 nm.

The treatments of the beads (in triplicate) were as follows:

| | |
|---|---|
| C− | Mix with Cy5-SS-dCTP and dCTP; no RT enzyme |
| C+ | Mix with Cy5-SS-dCTP and dCTP; with RT enzyme |
| U− | Mix with Cy5-SS-dUTP and dTTP; no RT enzyme |
| U+ | Mix with Cy5-SS-dUTP and dTTP; with RT enzyme |
| U+; cleave | As U+; followed by cleavage with DTT |
| U+; cleave; C+ | As ‘U+; cleave’ followed by incubation with C+ |

The reaction mixtures were as follows:

C− and C+: 0.4 μM Cy5-SS-dCTP; 1.6 μM dCTP; 40 U RNaseOUT (Invitrogen); 1× Reaction buffer as supplied with the RT enzyme (giving final concentrations of 50 mM Tris-HCl, pH 8.3 at room temperature, 75 mM KCl and 3 mM MgCl₂); 100 U Superscript II RNase H⁻ Reverse Transcriptase (Invitrogen) was included in C+.

U− and U+: 1.2 μM Cy5-SS-dUTP; 0.8 μM dTTP; 40 U RNaseOUT (Invitrogen); 1× Reaction buffer as supplied with the RT enzyme (giving final concentrations of 50 mM Tris-HCl, pH 8.3 at room temperature, 75 mM KCl and 3 mM MgCl₂); 100 U Superscript II RNase H³¹ Reverse Transcriptase (Invitrogen) was included in U+.

Cleave: 250 mM DTT in Washing Buffer.

A fluorescence control consisting of 200 μL TE was also included.

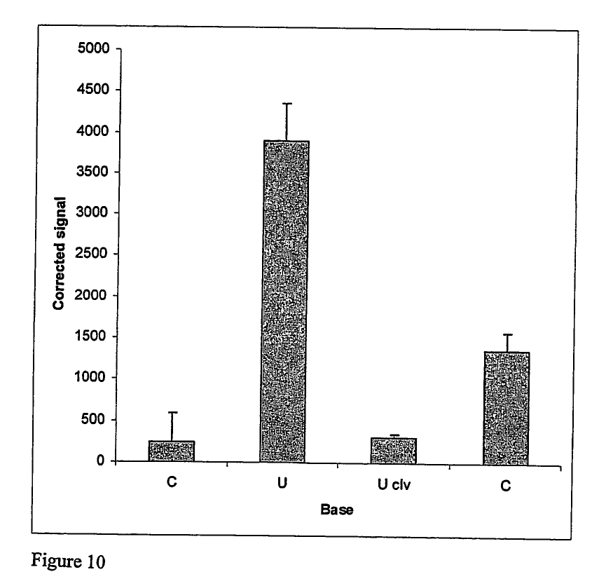

Fluorescence values were corrected using the relevant control values. The results are shown in FIG. 10. The correct sequence is TC. The data shows that the incorrect base, C, gave only a low signal whilst the correct base, U (equivalent to T) gave a high signal that could be removed by cleavage with DTT (‘U clv’). This was followed by incorporation of C, giving a clear signal over background that was greater than that obtained by the initial exposure to C.

### Example 6

### Selectivity Curve for Cy5-SS-dUTP/dTTP

This experiment was performed in essentially the same way as the example above. NUSPT-B (20 pmole) and E3PN19RNA (60 pmole) were annealed and immobilised as described in Example A. The equivalent of 1 pmole immobilised primer/template was transferred to wells of a filter plate. The primer was then extended using different mixtures of Cy5-SS-dUTP and dTTP in the presence of reverse transcriptase.

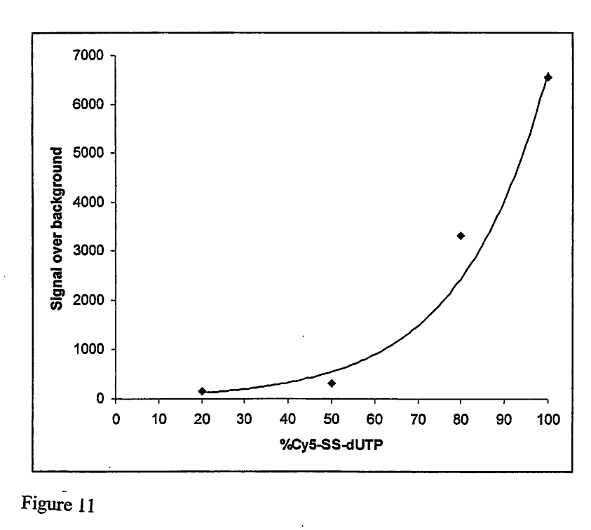

The enzyme was omitted in Controls. The beads were washed and transferred to a fluorimeter plate for measurement. The signals obtained in the presence of reverse transcriptase were corrected using the non-enzyme controls and plotted against the proportion of Cy5-SS-dUTP in the mixture (see FIG. 11). The results show a clear selectivity for the natural nucleotide, dTTP, over the labelled nucleotide Cy5-SS-dUTP.

### REFERENCES

Mazo A M et al. 1979. An improved rapid enzymatic method of RNA sequencing using chemical modification. Nucleic Acids Res 7(8):2469-82

Gibson C A et al. 1990. Primer extension dideoxy chain termination nucleotide sequencing of partially purified RNA virus genomes: a technique for investigating low titre viruses with extensive genome secondary structure. J Virological Methods, 29: 167-176

Donis-Keller H, A M Maxam, W Gilbert. 1977. Mapping adenines, guanines, and pyrimidines in RNA. Nucleic Acids Res 4 2527-38

Aoyama, H. 1989. Spermidine stimulates RNA-dependent reverse transcriptase activity. Biochem. Int. 19(1): 67-76

Peattie, D A. 1979. Direct chemical method for sequencing RNA. 1979. Proc. Natl. Acad. Sci. USA 76 1760-1764.

Sanger, F., G. S. Nickeln and A R Coulson 1977. DNA sequencing with chain terminating inhibitors. Proc. Natl. Acad. Sci. USA 74, 5463-5467.

Rocca-Serra, J. 1984. Dideoxy RNA sequencing: a rapid method for studying genetic information. Annales d'Immunologie 135 305-315

Bauer, G. J. 1990. RNA sequencing using fluorescent-labeled dideoxynucleotides and automated fluorescence detection. Nucleic Acids Res. 18 879-884.

Faulstich, K., K. Womer, H. Brill and J W Engels. 1997. A sequencing method for RNA oligonucleotides based on mass spectrometry. Anal. Chem. 69 4349-4353.

Agranovsky A. 1992. Exogenous primer-independent cDNA synthesis with commercial reverse transcriptase preparations on plant virus RNA templates. Anal. Biochem. 15; 203(1): 163-165.

Makeyev, E V, and D H Bamford. 2001. Primer-independent RNA sequencing with bacteriophage phi6 RNA polymerase and chain terminators. RNA 7 774-781.

Gerard, G. F. 1995. FOCUS (Invitrogen Corporation newsletter) 16, 102.

Sasaki N, Izawa M, Sugahara Y, Tanaka T, Watahiki M, Ozawa K, Ohara E, Funaki H, Yoneda Y, Matsuura S, Muramatsu M, Okazaki Y, Hayashizaki Y. 1998. Identification of stable RNA hairpins causing band compression in transcriptional sequencing and their elimination by use of inosine triphosphate. Gene 222 17-23

Kreader, C. A 1996. Relief of amplification inhibition in PCR with bovine serum albumin or T4 gene 32 protein. Appl. Environ. Microbiol. (62) 1102-1106.

Chandler, D. P., C. A. Wagnon and H Bolton Jr 1998. Reverse transcriptase(RT) inhibition of PCR at low concentrations of template and its implications for quantitative RT-PCR. Appl. Environ. Microbiol. (64) 669-677.

Villalva, C., C. Touriol, P. Seurat, P. Trempat, G. Delsol and P. Brousset. 2001. Increased yield of PCR products by addition of T4 gene 32 protein to the SMART PCR cDNA synthesis system. BioTechniques (31) 81-86.

Tanchou, V., C. Gabus, V Rogemond and J-L. Darlix 1995. J. Mol. Biol. 252 563.

Wadkins R M, C S Tung, P M Vallone, and A S Benight. 2000 The role of the loop in binding of an actinomycin D analog to hairpins formed by single-stranded DNA. Arch Biochem Biophys 384199-203

Klarman, G. J. C. A. Schauber, and B. D. Preston 1993. J. Biol. Chem. 268. 9793.

DeStefano, J. J. 1995. Arch. Virol. 140, 1775.

DeStefano, J. J. R. G. Buiser, L. M. Mallaber, T. W. Myers, R. A. Bambara, and P. J. Fay. 1991 J. Biol. Chem 266, 7423.

Chan, A. B. and J. D. Fox 1999. NASBA and other transcription-based amplification methods for research and diagnostic microbiology. Rev. Med. Microbiol. 10 185-196.

Gerard, G. F., R. J. Potter, M. D. Smith, K. Rosenthal, G. Dhariwal, J. Lee and D. K. Chatterjee. 2002. The role of template-primer in protection of reverse transcriptase from thermal inactivation. Nucleic Acids Res. (30) 3118-3129.

Compton, J. 1991.Nucleic acid sequence-based amplification. Nature (35) 91-92.

Kievits, T. B. Van Gemen, D. Van Strijp, R. Schukink, M. Dircks, H. Adriaanse, L. Malek, R. Sookanan, and P. Lens. 1991. NASBA isothermal enzymatic in vitro nucelic acid amplification optimised for the diagnosis of HIV-1 infection. J. Virol. Methods (35) 273-286.

Hill, C. S. 1996. Molecular diagnostics for infectious disease. J. Clin. Ligand Assay (19) 43-51.

Guatelli, J. C., K. M. Whitfield, D. Y. Kwoh, K. J. Barringer, D. D. Richman, and T. R. Gingeras. 1990. Isothermal, in vitro amplification of nucleic acids by a multienzyme reaction modelled after retroviral replication. Proc. Natl. Acad. Sci USA. (87) 1874-1878.

Loeb L A, Tartof K D, Travaglini E C. 1973. Copying natural RNAs with E. coli DNA polymerase I. Nat New Biol. Mar. 21, 1973;242(116):66-9.

Myers T W, Gelfand D H. 1991. Reverse transcription and DNA amplification by a Thermus thermophilus DNA polymerase. Biochemistry. Aug. 6, 1991;30(31):7661-6.

1. Method for the determination of the identity of at least one nucleotide in a RNA-molecule comprising the steps of:
(a) providing a single stranded form of the RNA-molecule;
(b) hybridising an oligonucleotide primer binding to a predetermined position of the RNA molecule;
(c) performing at least one primer extension reaction, whereby the oligonucleotide primer is extended on the RNA-molecule through incorporation of at least one nucleotide by the action of a RNA dependent polymerase;
(d) detecting the presence or absence of incorporation, thereby indicating the nucleotide identity of the RNA molecule in the relevant position;
whereby step (c) to (d) optionally are repeated.

2. Method according to claim 1, whereby step (c) to (d) are repeated.

3. Method according to claim 1 or 2, whereby the incorporated nucleotide(s) is (are) recorded.

4. Method according to claim 1-3, whereby the presence or absence of incorporation is indicated by the presence of a detectable moiety.

5. Method according to claim 4, wherein the detectable moiety is removed or neutralized in step (d) after the detection.

6. Method according to claim 1-5, whereby the primer extension reaction results in the release of a residue molecule.

7. Method according to claim 6, whereby the primer extension reaction results in the release of a PPi molecule only upon incorporation of a nucleotide.

8. Method according to claim 7, wherein step (c) is performed by including enzymes, comprising luciferase, apyrase, and ATP-sulfurylase, and reagents to detect the release of PPi to trigger the release of light.

9. Method according to claim 1-8, whereby at least one nucleotide is labelled, such as fluorescently of radioactively, thereby allowing the detection of step (c) to be performed by means of detecting the presence or absence of a labelled nucleotide.

10. Method according to claim 9, whereby the label on the labelled nucleotide is cleavable.

11. Method according to any one of the preceding claims, whereby the detection of step (c) is performed by means of detection of a change in physical properties of the RNA-molecule.

12. Method according to any one of the preceding claims, whereby the RNA dependent polymerase is an RNA dependent DNA polymerase or an RNA dependent RNA polymerase.

13. Method according to claim 12, whereby the RNA dependent RNA polymerase originates form any RNA virus or bacteriophage, such as bacteriophage phi 6.

14. Method according to claim 12, whereby the RNA dependent DNA polymerase is a RT-polymerase.

15. Method according to claim 14, whereby the RT polymerase is chosen from the group comprising: HIV-1 RT, M-MuLV RT, AMV RT, RAV2 RT, Thermoscript AMV RT, Superscript II M-MuLV RT, Tth DNA polymerase.

16. Method according to any one of the preceding claims, whereby a mixture of RNA dependent polymerases is added to the reaction mixture of step (a).

17. Method according to any one of the preceding claims, whereby the extension reaction is performed at a temperature ranging from 28 to 70° C.

18. Method according to any one of the preceding claims, whereby the pH of the extension reaction solution is in the interval from 7.6 to 8.6, preferably from 8.0 to 8.4.

19. Method according to any one of the preceding claims, whereby the concentration of deoxynucleotides is in the interval from 1 μM to 1 mM.

20. Method according to any one of the preceding claims, whereby the salt concentration of the reaction mixture is in the interval from 10 to 100 mM.

21. Method according to any one of the preceding claims, wherein the oligonucleotide primer is a DNA primer.

22. Method according to claim 21, whereby the nucleotide is the deoxynucleotide dATP, which further is exchanged for the analogue alpha-S-dATP.

23. Method according to claim 1-20, wherein the oligonucleotide primer is a RNA primer.

24. Method according to claim 23, whereby the nucleotide ATP is exchanged for the analogue alpha-S-ATP

25. Method according to any one of the preceding claims, whereby a RNA-secondary structure reducing reagent, preferably chosen from the group comprising T4 Gene 32 Protein, retroviral nucleocapsid protein, actinomycin D, glycerol, methyl mercury hydroxide, methoxyamine-bisulfite, DMSO, spermidine, formamide, SSB (single stranded binding protein) and blocking primer, is included in the extension reaction.

26. Method according to any one of the preceding claims, whereby the RNA molecule is subjected to an RNA amplification prior to the extension reaction.

27. Method according to claim 26, whereby the nucleotide rITP is exchanged for rGTP in the amplification.

28. Method according to any one of the preceding claims, whereby the RT polymerase essentially lacks RNase H activity.

29. Method according to any one of the preceding claims, wherein the oligonucleotide primer is immobilised to a solid phase or wherein the RNA molecule is captured to a solid phase by an immobilised oligonucleotide.

30. Method according to any one of the preceding claims, whereby the quantity of the RNA-molecule is determined by measuring the intensity of the incorporation signal and comparing it to a reference.

31. Kit for performing the nucleotide identification of claim 1-30, comprising in separate vials a RNA dependent polymerase, nucleotides, necessary enzymes for a sequencing-by-synthesis reaction, and optionally other necessary reagents.

32. Kit according to claim 31, which further comprises a RNA quantity reference sample.

33. Method for determining the sequence of a ribonucleic acid molecule comprising the steps of;
a) providing a single-stranded form of said ribonucleic acid molecule;
b) hybridizing a primer to said single stranded form of said ribonucleic acid molecule to form a template/primer complex;
c) enzymatically extending the primer by the addition of an RNA dependent polymerase and a mixture of nucleotides and a derivative of said nucleotides, wherein the derivative of said nucleotide comprises a label linked to a nucleotide via an optionally cleavable link and wherein the proportion in the mixture between the nucleotides and the derivative of said nucleotide is within the range of 1-60%, 1-50%, 1-40%, 1-30%, or 1-20%, preferably in the range of 5-60%, 5-50%, 5-40%, 5-30%, or 5-20%, or more preferably in the range of 10-60%, 10-50%, 10-40%, 10-30%, or 10-20%.
d) determining the type of nucleotide added to the primer;

34. Method according to claim 33, wherein the label is neutralized after step d) by the addition of a label-interacting agent or by bleaching, preferably by photobleaching.

35. Kit comprising, in separate compartments, a mixture of natural nucleotides and a derivative of said nucleotides according to step c) of claim 33, and at least one of the following components; an RNA dependent polymerase, a reducing agent, a carrier, a capping agent, an apyrase, an alkaline phosphatase, a PP-ase, a single strand binding protein or the protein of Gene 32, for performing the method according to claim 33-34.


SEQUENCE LISTING (7 sequences)

SEQ ID NO: 1 - DNA, 35 bp, Artificial
  Artificial Primer
ctggaattcgtctgaactggccgtcgttttacaac

SEQ ID NO: 2 - RNA, 35 nt, Artificial
  Artificial Primer
cuggaauucgucugaacuggccgucguuuuacaac

SEQ ID NO: 3 - DNA, 17 bp, Artificial
  Artificial Primer
gtaaaacgacggccagt

SEQ ID NO: 4 - DNA, 16 bp, Artificial
  Artificial Primer
tcagacgaattccagc

SEQ ID NO: 5 - DNA, 26 bp, Artificial
  Artificial Primer
gtaaaacgacggccagtucagacgaa

SEQ ID NO: 6 - DNA, 36 bp, Artificial
  Artificial Primer
caacattttgctgccggtcaagtctgcttaaggtcg

SEQ ID NO: 7 - DNA, 17 bp, Artificial
  Artificial Primer
gtaaaacgacggccagt